(19) United States
(12) Patent Application Publication
(10) Pub. No.: US 2014/0343672 A1
(43) Pub. Date: Nov. 20, 2014

(54) ARTIFICIAL HEART

(71) Applicant: Stavros Kargakis, Raeren (BE)

(72) Inventor: Stavros Kargakis, Raeren (BE)

(21) Appl. No.: 14/262,028

(22) Filed: Apr. 25, 2014

Related U.S. Application Data
(63) Continuation of application No. PCT/EP2012/071011, filed on Oct. 24, 2012.

(30) Foreign Application Priority Data
Oct. 25, 2011 (DE) 102011054768.1

(51) Int. Cl. A61M 1/10; A61M 1/12

(52) U.S. Cl. CPC A61M 1/1051; A61M 1/1005; A61M 1/1081; A61M 1/1096; A61M 1/127; A61M 2205/04; A61M 2205/106; A61M 2206/20; A61M 2210/125; A61M 2205/0216
USPC 623/3.19

(57) ABSTRACT

An artificial heart, comprising an energy storage device, an actuator device and two pumping chambers, each with an inlet opening and an outlet opening, wherein each pumping chamber is bounded by at least one flexible wall and one rigid wall and the volume of each pumping chamber can is variable by deformation of the flexible wall by means of the actuator device, whereby the flexible wall, starting from a suction position in which the volume of the respective pumping chamber is at a maximum, can be converted to a pumping position in which the volume of the feed chamber is at a minimum by means of the actuator device, wherein the inlet opening and the outlet opening of each pumping chamber each have a non-return valve and the non-return valves can be reciprocally in an opened and a closed position, wherein the actuator device is located between the pumping chambers.

### RELATED APPLICATIONS

This application is a continuation of International application PCT/EP2012/071011 filed on Oct. 24, 2912 which claims priority from German patent application 10 2011 054 768.1 filed on Oct. 25, 2011, both of which are incorporated in their entirety by this reference.

### FIELD OF THE INVENTION

The invention relates to an artificial heart.

### BACKGROUND OF THE INVENTION

Artificial hearts of the general type similar to the one described supra are known in the art though they are still not fully developed from an engineering point of view and therefore are rarely used in practical applications.

In particular the European patent document EP 0 971 756 B1 discloses an artificial heart which includes two artificial heart chambers or ventricles arranged in a V-shape which are each respectively actuation by a motor pump unit, wherein the V formed by the heart chambers is turned upside down, thus stands on its head. The two artificial heart chambers are respectively operated by a membrane unit which is deformed through one of the two respectively associated motor pump units, wherein a volume of the respective heart chamber changes between a maximum and a minimum during operation of the heart. The membrane units of the heart chambers are controlled through a hydraulic pressure, wherein the motor pump units load a fluid with pressure that is arranged in a respective pressure chamber and that is arranged on a side of the respective membrane unit which side is oriented away from the respective heart chamber. These way surfaces of the membrane units can be deformed by applying a uniform pressure.

Besides energy storage device the entire control electronics and mechanisms of the artificial heart are enclosed within a rigid housing, wherein the housing can be connected with a blood circulatory system through a total of four channels. Two respective channels of the four channels are respectively connected with one of the two hear chambers and simulate incase of the right heart chamber the hollow vein(s) and the lung artery or incase of the left heart chamber the lung vein(s) and the aorta.

A downside of the device illustrated in EP 0 971 756 B1 is the relatively large space requirement and the relatively high weight of the heart compared with a natural heart. Furthermore the “connections” for connecting the heart with the blood circulatory system of the patient are primarily adapted to the configuration of the artificial heart substantially differ with respect to their positions from a natural heart, so that a connection of the blood vessels during implantation of the artificial heart is rather complex.

Another embodiment of the artificial heart in which the two heart chambers are arranged in a V-shape relative to one another is illustrated in EP 0 079 373 B, wherein an orientation of the V in this case is right side up, thus the V is not standing on its head. With respect to its basic function the artificial heart invented by applicant is similar to the document recited supra, but the arrangement of the pump units is different. Thus these are two pump units, wherein one respective pump unit is designated for operating one heart chamber. The heart chambers are defined by a flexible membrane on one side which is deformable by the hydraulic pressure of a fluid which is generated by the pump unit. The pump units are thus arranged below the heart chambers, whereas they are arranged laterally adjacent to the heart chambers in the document EP 0 971 756 B1 recited supra.

Also this device predominantly has the problem that the space requirement significantly exceeds the space requirement of a natural heart and thus its functionality might suffice but there might be problems in practical applications.

Another artificial heart is disclosed in EP 0 324 669 B1, wherein a difference of the heart illustrated herein compared to the documents recited supra is primarily the arrangement of the actuation devices which are respectively formed per heart chamber by an electromechanical device in combination with a respectively associated micro-pump. With respect to the space requirement and the shape of the artificial heart the disembodiment is even worse than the two embodiments recited supra since the actuation devices are respectively arranged approximately perpendicular to a center plane of the heart chambers arranged in a V-shape and the actuation devices protrude from the heart chambers into the body of the patient. Furthermore the heart illustrated in this document has a disadvantage in that is uses a compensation cavity that is external, this means arranged outside of an envelope that encloses the heart, wherein the compensation cavity requires additional space within the body of the patient and causes additional complexity when connecting it with the heart.

Furthermore DE 23 37 497 B2 illustrates an artificial heart in which the atriums of the heart are depicted in addition to the heart chambers and also activated through proprietary membranes. This is preformed through alternating switching of a heart chamber and the respective associated atrium, wherein a membrane associated with the atrium conducts the blood flow from the atrium into the heart chamber, wherein the membrane which is associated with the heart chamber pumps the blood flow into the aorta or the lung artery depending on which of the heart chambers (left or right it is). Generating a hydraulic pressure in a transmission fluid which in turn impacts the membranes is generated through two actuation devices, wherein a respective actuation device is associated with a side of the heart, wherein a pump piston of a respective actuation device is moved through magnetism. The disclosed heart is disadvantageous besides the high weight and the high space requirement in particular because the illustrated arrangement always generates zero flow areas in the blood stream which eventually contribute to coagulation of blood in the blood flow.

An artificial heart is furthermore disclosed by DE 24 49 320 in which a actuation device is arranged between the heart chambers. This has the advantage that both heart chambers can be actuated with a single actuation device, wherein movable wall elements of the heart chambers can be pressed “outward” through the actuation device in a direction that is oriented away from the actuation device which reduces a volume of a respective heart chamber. A reset of the movable wall elements into the original position is performed by reset springs. The disadvantages of this artificial heart are the same of the ones in DE 23 32 497 B2.

### BRIEF SUMMARY OF THE INVENTION

The present invention relates to providing an artificial heart which is characterized by exterior dimensions, mass and functionality very similar to a natural heart and which is therefore implantable into a pericardial cavity.

The object is achieved through a simulation of a human heart, including an energy storage device, an actuation device as well as two pumping chambers, respectively including an inlet opening and an outlet opening, wherein each pumping chamber is defined by at least one flexible wall and one rigid wall and a volume of each pumping chamber can be varied by a deformation of the flexible wall through the actuation device, in that the flexible wall, starting from a suction position in which the volume of the respective pumping chamber is at a maximum, can be transferred into a pumping position in which the volume of the storage chamber is at a minimum through the actuation device, wherein the inlet opening and the outlet opening of each pumping chamber respectively include a non-return valve and the non-return valves can be brought alternatively into an open position and into a closed position, whereby a directed flow of blood from the respective inlet opening to the respective outlet opening is generateable, wherein the actuation device is arranged between the pumping chambers.

Improving upon a known artificial heart the object is achieved according to the invention in that the pumping chambers are configured elongated, wherein a center line of a respective pumping chamber is cambered and advantageously has a camber radius between 10 cm and 40 cm, further advantageously between 15 cm and 30 cm and a cross-section of the pumping chamber orthogonal to the center line of the respective pumping chamber has a form of an ellipse, wherein a ratio between a semi-major axis and a semi-minor axis of the ellipse is between 1.0 and 2.0, advantageously between 1.1 and 1.5.

The described shape of the pumping chambers can also be designated as “banana shape”, “kidney shape” or “sickle shape”, wherein the pumping chambers advantageously have a length of at least 10 cm, wherein the semi-major axis has at least a length of 1.0 cm and the semi-minor axis has a length of at least 0.5 cm. The length of the pumping chambers corresponds to a length of a straight line connecting both ends of the respective pumping chamber, this means the length of a pumping chamber is not identical with a length of its cambered center line. Instead the center line of a pumping chamber designates a circular arc, wherein the length of the pumping chamber corresponds to the length of a secant connecting the ends of the circular arc. The dimensions of length, semi-major axis and semi-minor axis are thus always matched so that the respective pumping chamber has a volume between 60 cm³ and 120 cm³, advantageously a volume of 80 cm³. The center line of a pumping chamber is interpreted as a connecting line of centers of gravity of all cross sections through the pumping chamber.

Experiments have shown that the described shape of the pumping chambers is particularly advantageous for blood transportation, wherein the flexible walls of the pumping chambers in this sense apply particularly well to a rigid wall so that no zero flow velocity areas are generated. The risk of blood clotting is therefore not provided for the artificial heart according to the invention.

In an advantageous embodiment of the heart, an inner cavity of a respective pumping chamber is defined by an elastic envelope, wherein the elastic envelope forms the rigid wall of the pumping chamber in a support portion oriented away from the actuation device, advantageously over an entire length of the pumping chamber through interaction with a rigid support element that has a U-shaped cross section and wherein the elastic envelope forms the flexible wall outside of the support portion, wherein half of a circumference of the elastic envelope interacts with the U-shaped support element advantageously in a cross section through the elastic envelope orthogonal to a center line of the respective pumping chamber. This means that the rigid wall and the flexible wall are not respectively formed by themselves by a particular element, but the entire pumping chamber is so to speak formed by a flexible wall, wherein the flexible wall performs the function of a rigid wall solely through the interaction with the rigid support element. At places where the elastic envelope is not supported through the support element the elastic envelope is flexible and elastically deformable and can apply to the rigid wall that is stiffened by the support element.

In a particularly advantageous embodiment of this combination of rigid support element and elastic envelope the elastic envelope and the support element are connected with one another through form locking in force transferring manner, wherein the support element advantageously includes at least one under cut over its entire length, wherein a corresponding key of the elastic envelope engages the undercut groove. Advantageously the support element includes several of the grooves and analogously the elastic envelope has several respective keys so that the connection of both components is particularly reliable. Alternatively or in addition to the described variant and also a friction locking connection between both elements is conceivable, wherein in particular a flat glue joint of the support element with the envelope can be advantageous. A combination of a form locking connection and a friction locking connection between the support element and the envelope can be particularly advantageous.

The pumping chambers which are intended to simulate the ventricles of a natural heart are advantageously arranged adjacent to one another according to the invention, wherein according to the prior art a V-shaped or inverted V-shaped arrangement of both heart ventricles is very common thus in addition to the offset arrangement of both pumping chambers a relative rotation of both pumping chambers is provided. A particular advantage of the artificial heart according to the invention is that the pumping chambers are arranged parallel to one another, wherein a parallel arrangement means that two planes respectively associated with a pumping chamber are arranged parallel to one another, wherein the planes contact their respective pumping chambers tangentially and respectively extend through a high point of the associated pumping chamber, wherein the high point is arranged in the rigid wall of the respective pumping chamber and is defined with respect to its position so that incase the flexible wall is in its suction position a distance between the flexible wall and the rigid wall measured orthogonal to the rigid wall is at a maximum. Compared with the V-shaped arrangement a space between the pumping chambers for the parallel arrangement of the type is greater and therefore much better useable. Furthermore the parallel arrangement of the pumping chambers yields advantages for driving the flexible walls of the pumping chambers as will be separately described infra.

In order to protect the entire artificial heart an envelope should be provided which completely encloses both pumping chambers and the actuation device. An envelope of this type can be made of a rigid material, for example a coated plastic material, so that mechanical impacts which impact patients from an outside can be absorbed so that the heart is protected against damages. Besides arranging the pumping chambers and the actuation device furthermore the energy storage device from which eventually the actuation device is supplied energy can be advantageously arranged within the envelope so that a separate wiring of the heart with an external can be completely omitted. In view of the current storage techniques for storing electrical energy an arrangement of the energy storage device outside of the envelope, however, has the great advantageous that a replacement of the energy storage device can be performed in a much simpler manner, for example the energy storage device is placed easily reachable proximal to the skin surface of the patient. In case large storage capacities can be implemented an integral arrangement of the energy storage device, this means an arrangement of the energy storage device in the interior of the envelope and also of the remaining elements of the heart is particularly advantageous.

An artificial heart according to the invention is particularly advantageous in which for one of the two pumping chambers an arrangement of the associated outlet opening corresponds to a natural arrangement of a lung artery of a right heart chamber of the human heart. Advantageously this is the right pumping chamber in analogy to a right heart chamber for a natural heart. The position of the outlet opening close to a natural position is particularly advantageous for implanting the artificial heart since a complex coupling of the artificial heart to the natural lung artery can be omitted. This saves time during implantation and provides a lower treatment risk for the patient and more efficient operations for the treating physician.

This advantage is analogously transferable to the aorta with respect to the left pumping chamber. For one of the two pumping chambers accordingly the outlet opening should correspond to the natural position of the aorta.

For further approximation of the basic anatomy of the artificial heart to the natural heart furthermore a connection cavity should be arranged at one of the two pumping chambers on a side of the associated inlet opening which side is oriented away from the pumping chamber, wherein preferably two conduits for conducting a blood flow lead into the connecting cavity. The connecting cavity is connected through the inlet opening with the respective pumping chamber, wherein the non-return valve described supra is arranged in the inlet opening. Thus, the connecting cavity is used for joining separate blood circuits. The non-return valve is advantageously arranged using a clip system so that the non-return valve is arranged in a removable manner in the respective in let opening or outlet opening between the connecting cavity and the pumping chamber so that the non-return valve is replaceable in a particularly simple manner. Through an arrangement of the described pumping chamber which includes the described connecting cavity in a manner that it is being used as a replacement for the right heart chamber accordingly a connection of the artificial heart to the hollow veins is possible in a particularly simple manner. Optimally the pumping chamber which includes the described connecting cavity with two conduits leading into the connecting cavity simultaneously forms the same pumping chamber whose position is approximated to the position of the associated outlet opening is approximated to the position of the lung artery in a natural heart. This way the pumping chamber is optimally adapted to the geometry of the right heart chamber of the natural heart, so that it is particularly simple to implant the heart into a patient while maintaining the natural blood vessels.

Analogously it is particularly advantageous when a connecting cavity is arranged at one of the two pumping chambers on a side of the associated inlet opening which side is oriented away from the respective pumping chamber wherein preferably four conduits for conducting a blood flow lead into the connecting cavity. According to the explanations provided supra regarding using a pumping chamber of the heart according to the invention as a replacement for the right heart chamber of the natural heart accordingly the pumping chamber which provides a connecting cavity with four conduits leading into the connecting cavity is suited particularly well for replacing the left heart chamber of the natural heart since the four conduits can simulate the four lung veins of the natural heart. Analogously the pumping chamber is advantageously the pumping chamber in which the outlet opening according to the preceding description is imaged according to the arrangement of an aorta for a natural heart.

Independently from a position of the connections or the inlet and outlet openings of the pumping chambers an artificial heart according to the invention is operable in a particularly advantageous manner when at least one flexible wall of at least one pumping chamber is moved by at least one transmission device, preferably by a plurality of transmission devices, wherein the transmission device is actuated by the actuation device. Transmission devices in this case are all devices through which a force generated by the actuation device is transferable to the flexible wall.

A particularly advantageous embodiment is the configuration of the transmission device as a piston-cylinder unit. The piston-cylinder unit can be actuated by the actuation device in that the piston cylinder unit builds up a pressure within the cylinder of the cylinder piston unit. The pressure starting from a starting position of the piston cylinder unit impacts the walls of the cylinder and also the bottom side of the piston which is then pressed out of the cylinder. The piston is eventually connected with a flexible wall of the pumping chamber so that a movement of the piston simultaneously leads to a movement or deformation of the flexible wall. Thus, the flexible wall can be transferred from the suction position into the pumping position, wherein the pumping position of the flexible wall corresponds with an end position of the piston cylinder unit; this means that the piston of the piston the cylinder unit in this end position is moved by a maximum amount out of the cylinder. By using a transmission device of this type the volume of the associated pumping chamber can be cyclically changed between a maximum and a minimum, so that eventually blood is continuously displaced out of the pumping chamber or sucked in again and a pulsating blood flow can be eventually generated this way.

As an alternative to this arrangement a purely mechanical actuation mode is conceivable in which the transmission device is formed by a cam-tappet unit. Thus, at least one cam is actuated by the actuation device, wherein the cam is connected with at least one tappet which performs a lifting movement through a rotation of the cam. In analogy to the previously described piston the tappet should eventually be connected with the flexible wall of a pumping chamber so that the pumping chamber is deformable by the tappet.

Using a cam-tappet unit can be advantageous in particular when the cam is configured so that it has a plurality of peaks and valleys so that an associated tappet performs a plurality of strokes during one a revolution of the cam. This way the artificial heart according to the invention can be used in a particularly energy-saving manner, since already a slow speed of revolution of the cam facilitates performing a plurality of pump cycles or heartbeats so that a pulsating blood flow is generateable.

Furthermore using a cam-tappet unit has the particular advantage that a cam can drive plural tappets, in particular two tappets simultaneously. Through the arrangement of the actuation device between the pumping chambers two pumping chambers can be operated simultaneously through a transmission device.

As already stated supra the artificial heart shall be optimally configured with a plurality of transmission devices, wherein a plurality of transmission devices per pumping chamber or per flexible wall shall be provided. This is advantageous in particular because the flexible walls of a respective pumping chamber, in case of loading a limited surface with a force, is only locally deformed and locations of the flexible wall that are further away from a location were the force is introduced remain non deformed and thus do not contribute to displacing blood out of the pumping chamber. In particular a standstill of the blood has to be prevented at all cost since as already described supra a non-continuous flow of the blood leads to clotting of the blood which can be a clear and imminent danger for the health of the patient. Through a force introduction that is distributed over the flexible wall through a plurality of transmission devices this can be excluded so that no portions within the respective pumping chamber remain in which the blood is not pumped anymore. For the actual advantageous embodiments of the transmission device configured as a piston cylinder unit or a cam-tappet unit this means that a plurality of pistons or tappets should engage respectively in a distribution over the flexible walls of both pumping chambers that is as homogeneous as possible, so that the walls deform in a very even manner.

The problem of introducing a force into the flexible walls of the pumping chambers in a very even manner can be solved absent the described transmission devices in a particularly simple manner through loading the flexible walls with a hydraulic pressure. The artificial heart thus shall be configured accordingly so that at least one of the flexible walls of at least one pumping chamber is movable through a hydraulic pressure, wherein the wall is loadable with a transmission fluid advantageously on a side that is oriented away from the pumping chamber wherein the transmission fluid is loadable with a pressure through an actuation device, wherein the actuation device includes a pump. The transmission fluid in this arrangement should be arranged in pressure cavities which are respectively arranged on a side of the flexible wall that is oriented away from the respective pumping chamber and wherein the pressure cavities are connected with the pump of the actuation device so that the transmission fluid is loadable with a pressure. Advantageously a single pump should be used to control the transmission fluid in both pressure cavities simultaneously, so that a simultaneous transfer of the flexible walls of both pumping chambers from a suction position into a pumping position is provided in a particularly simple manner. Furthermore a simultaneous pressure drop facilitates a simultaneous reversal movement of the flexible walls from the pumping position into the suction position in a particularly simple manner.

It is advantageous when a second actuation device is provided besides the actual actuation device, wherein the second actuation device, in case there is a defect in the first actuation device, can maintain the function of the artificial heart at least over a particular time period, so that there is an opportunity to replace or repair the first actuation device.

Through a deformation of the flexible walls of the pumping chambers through a hydrostatic pressure an even deformation of the locations of the flexible wall which are in contact with the transmission fluid is facilitated. The problem of local deformation is thus solved.

Optimally the flexible walls should be in contact with the transmission fluid over their entire surface and shall be loadable over their entire surface with the hydraulic pressure. This way a formation of zero flow velocity area in the pumping chambers is effectively prevented.

With respect to an embodiment of the no return valves in particular using a flap element is advantageous, wherein the flap element advantageously includes a plurality of flap segments through which a flow of the blood in a closing direction of the flap element is cut off and vice versa a flow of the blood in an opening direction of the flap element is released. The individual flap segments of a respective flap element are thus configured so that they are evenly distributed over a circumference of a wall of a respective controlling opening cross-section of the associated conduit and are flappable into an opening position starting from a orientation that is perpendicular to the flow direction in which orientation they overlap and stop a fluid flow, wherein the fluid flow is released in the opening position. The flap segments are thus configured so that they cannot flap through into a direction in which the fluid flow shall be prevented and thus do not release the fluid flow involuntarily.

Eventually an artificial heart of this type is particularly advantageous in which at least one of the flexible walls has individual portions which are deformable in a time offset manner through an actuation device while transferring the entire wall from a suction position into a pumping position. A time offset control of the deformation of a respective flexible wall can be implemented in particular using a transmission device as described supra in that for example pistons and tappets arranged at various locations of the respective flexible wall are actuated with a time in a time offset and thus individual portions of the flexible wall move towards the rigid wall earlier and already displace blood before this is the case in portions in which a respective transmission device has not yet been activated. For a control of the flexible wall through hydraulic pressure, however, this method does not work.

The time offset deformation or the time offset displacement of the blood from the respective pumping chamber associated therewith can be used in particular to control the blood flow out of the pumping chamber in a direction towards a desired location of the pumping chamber. For example when the outlet opening of a pumping chamber is arranged in an upper portion thereof, it is advantageous to displace the blood starting from a lower portion of the pumping chamber in order to generate a peristaltic pumping of the blood within the pumping chamber in this manner.

### BRIEF DESCRIPTION OF THE DRAWINGS

The invention artificial heart according to the invention is now described in more detail based on an embodiment with reference to drawing figures, wherein:

FIG. 1 illustrates a sectional view of an artificial heart according to the invention;

FIG. 2 illustrates a sectional view through another artificial heart according to the invention;

FIG. 3 illustrates a sectional view through another artificial heart according to the invention;

FIG. 4 illustrates the same embodiment as FIG. 3 in another cross sectional view;

FIG. 5a illustrates a sectional view of pumping chamber in its suction position;

FIG. 5b illustrates the same sectional view as 5a, but in a pumping position; and

FIG. 6a through 6c illustrates details of a flap element that functions as a no return valve.

### DETAILED DESCRIPTION OF THE INVENTION

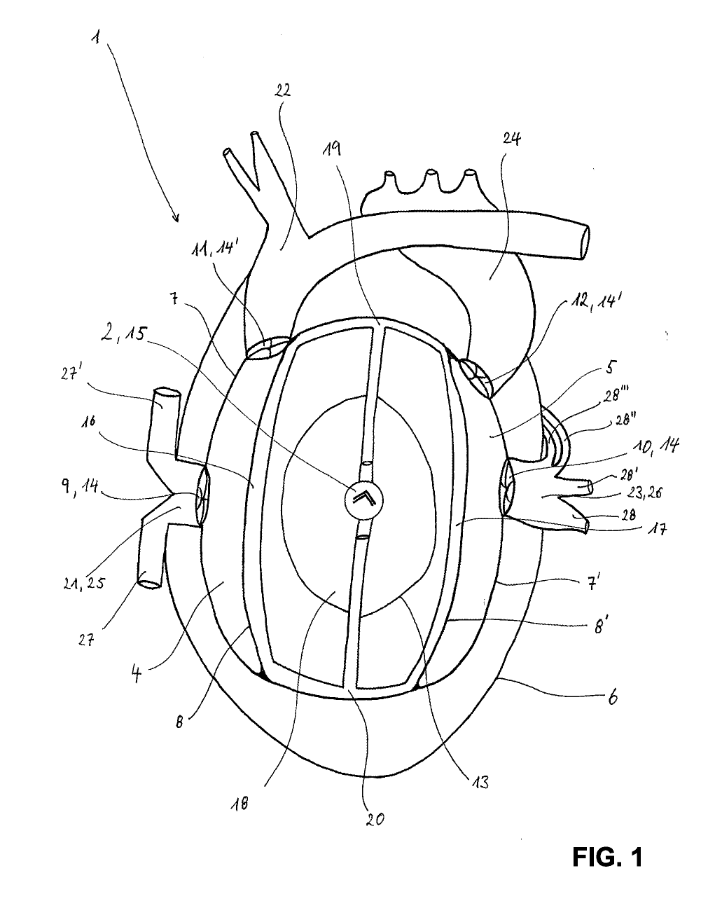

A first embodiment that is illustrated in FIG. 1 depicts an artificial heart 1 with an actuation device 2, an energy storage device 3 which is not illustrated and two pumping chambers 4, 5, wherein a right pumping chamber 4 is arranged on a left side of the illustrated heart 1 and a left pumping chamber 5 is arranged a right side of the illustrated heart 1. The heart 1 is enclosed by an envelope 6 and thus forms a closed unit. In the interior of the envelope 6 a control unit which is not illustrated is arranged in addition to the recited components. The control unit among other things is used for regulating a beat frequency of the heart 1. Thus for example the beat frequency can be increased under a physical load and can be regulated down in a relaxation phase of the body of a patient to a resting pulse or a resting frequency. The envelope 6 itself is thus preferably made from a biocompatible plastic material that has damping properties. Thus, for example a silicon elastomeric material can be used whose surface is provided with a biocompatible coating.

The pumping chambers 4, 5 respectively include a rigid wall 7, 7′ and a flexible wall 8, 8′, wherein a volume of each pumping chamber 4, 5 is adjustable by deforming the respective flexible wall 8, 8′. In FIG. 1 both walls 8, 8′ are depicted in a suction position in which a volume of both pumping cavities 4, 5 is at a maximum. The recited deformation of the flexible wall 8, 8′ in a direction towards the respective associated rigid wall 7, 7′ facilitates reducing the volume of the respective pumping chamber 4, 5 until the flexible wall 8, 8′ eventually arrives at its pumping position in which the volume of the respective pumping chamber 4, 5 is at a minimum. Optimally the volume of the pumping chambers 4, 5 is reduced to almost zero during a transition of the flexible walls 8, 8′ from their suction position into a pumping position. The illustrated pumping chambers 4, 5 respectively have a volume of 80 cm³.

Both pumping chambers 4, 5 respectively include an inlet opening 9, 10 and an outlet opening 11, 12 wherein the right pumping chamber 4 includes the inlet opening 9 and the outlet opening 11 and the left pumping chamber 5 includes the inlet opening 10 and the outlet opening 12. All inlet openings 9, 10 and outlet openings 11, 12 respectively include a non return valve configured as a flap element 14, 14′, wherein the flap element 14 is arranged at the inlet openings 9, 10 so that a blood flow is conductible exclusively one direction oriented into the respective pumping chamber 4, 5 and inversely the flap elements 14′ are arranged at the outlet openings 11, 12 so that a blood flow is conductible exclusively in a direction out of the respective pumping chambers 4, 5.

Through a change in the volume of the pumping chambers 4, 5 through the flexible walls 8, 8′ which are actuated by the actuation device 2 blood disposed in the pumping chambers 4, 5 is moved or pumped. In case of transitioning the flexible walls 8, 8′ from the pumping position into the suction position, a vacuum is generated within the associated pumping chambers 4, 5, wherein the vacuum causes blood arranged outside the pumping chambers 4, 5 to be sucked in. Due to the non return valves configured as flap elements 14 blood can only enter through the respective inlet openings 9, 10 into the associated pumping chambers 4, 5. A blood flow directed into the pumping chambers 4, 5 through the outlet openings 11, 12 is, however, blocked through the flap elements 14′. During a subsequent transfer of the flexible walls 8, 8′ from the suction position into the pumping position, a pressure is inversely built up in the respective pumping chambers which cause the blood to be displaced from an interior of the pumping chambers 4, 5. Due to the flap elements 14 a run out of the blood, however, is only feasible through the outlet openings 11, 12, the inlet openings 9, 10, however, are closed. Through this method eventually an oriented blood flow can be generated through the heart 1 which is similar to a natural heart.

In the embodiment illustrated in FIG. 1 the flexible walls 8, 8′ are actuated by a hydraulic pressure of a transmission fluid. This pressure is generated by a pump 15 which is a component of the actuation device 2. The pump 15 is arranged between a compensation cavity 18 and two operating cavities 16, 17 wherein the operating cavity 16 is arranged on a side of the flexible wall 8 which side is oriented away from the pumping chamber 4 and the operating cavity 17 is arranged on a side of the flexible wall 8′ which side is oriented away from the pumping chamber 5. Both operating cavities 16, 17 are connected with one another so that the transmission fluid in both operating cavities 16, 17 is always at the same pressure level. When the flexible walls 8, 8′ are in the suction position a volume of the operating cavity 16, 17 is minimized, whereas a volume of the compensation space 18 which is enclosed by an elastic wall 13 is maximized wherein all volumes of the operating cavities 16, 17 and of the compensation cavity are completely filled with the transmission fluid.

Based on the suction position of the two flexible walls 8, 8′ illustrated in FIG. 1 a transfer of the flexible walls into their pumping position is achieved in that the pump 15 pumps the transmission fluid arranged in the compensation cavity 18 into the operating cavities 16, 17 so that a pressure upon the surfaces of the flexible walls 8, 8′ is built up. The flexible walls eventually deform under the pressure and respectively move towards the associated rigid walls 7, 7′ and thus displace the blood from the pumping chambers 4, 5. In order to move the flexible walls 8, 8′ completely into their pumping positions the volumes of the two operating cavities 16, 17 have to increase by the same amount by which the pumping cavities 4, 5 are reduced in volume. Consequently the compensation cavity 18 has to have at least a volume which corresponds to the sum of the volumes of the pumping chambers 4, 5. During movement of the flexible walls 8, 8′ from their suction position into their pumping position the compensation cavity 18 is typically approximately emptied while the operating cavities 16, 17 are analogously filled with the transmission fluid.

During a reverse transfer of the flexible walls 8, 8′ from the pumping position into the suction position the transmission fluid is accordingly pumped from the operating cavities 16, 17 back again into the compensation cavity 18. This process generates a vacuum on a side of the flexible walls 8, 8′ which side is oriented away from the pumping chambers 4, 5 wherein the vacuum eventually causes a tension force over the surface of the walls 8, 8′, wherein the tension force moves the walls 8, 8′ away again from the respective associated rigid walls 7, 7′. This movement of the flexible walls 8, 8′ from the pumping position into the suction position is optimally supported by a reset force of the walls 8, 8′ which results from an elastic deformation of the walls 8, 8′ wherein the walls 8, 8′ in their suction position are approximately without tension and in their pumping position they are deflected by a maximum amount and thus under tension. Conduits 19, 20 through which the pump 15 is connected with the operating cavities 16, 17 are provided with valves for both movement directions of the flexible walls 8, 8′ so that the transmission fluid is pumped out of the compensation space 18 for displacing the blood from the pumping chambers 4, 5 and vice versa while maintaining a pumping direction of the pump 15 the transmission fluid is pumped out of the operating cavities 16, 17 into the compensation cavity 18.

The inlet openings 9, 10 and the outlet openings 11, 12 of the pumping chambers 4, 5 are analogously to a natural heart provided with conduits 21, 22, 23, 24 which are connected with natural blood vessels of a human so that individual conduits 21, 22, 23, 24 communicate with individual natural blood vessels. In case of the right pumping chamber 4 this means that the conduit 21 arranged at a side of the inlet opening 9, which side is oriented away from the pumping chamber 4, corresponds to the hollow veins of the human, or is connected therewith and the conduit 22 arranged on a side of the outlet opening 11 which side is oriented away from the pumping chamber 4 corresponds to a lung artery of the human or is connected with the lung artery of the human. In analogy thereto the conduits 23, 24 of the left pumping chamber 5 correspond to the lung veins or the aorta of the human.

The conduits 21, 23 which are respectively connected with the inlet openings 9, 10 of the two pumping chambers 4, 5, differently from the conduits 22, 24 respectively include a connecting cavity 25, 26, wherein a plurality of small conduits 27, 27′, 28, 28′, 28″, 28′″ are connected with the connecting cavities 25, 26. Thus, the conduits 27, 27′ are associated with the connecting cavity 25 which communicates in turn with the connecting cavity 9 and thus with the right pumping chamber 4 and the conduits 28, 28′, 28″, 28′″ are connected with the connecting cavity 26, which communicates with the inlet opening 10 and thus with the left pumping chamber 5. The conduits 27, 27′ are thus associated with two hollow veins, whereas the conduits 28, 28′, 28″, 28′″ correspond to four lung veins. The blood from all conduits 27, 27′, 28, 28′, 28″, 28′″ collects in the respectively associated connecting cavities 25, 26 before it is pumped through the associated inlet openings 9, 10 into the pumping chambers 4, 5.

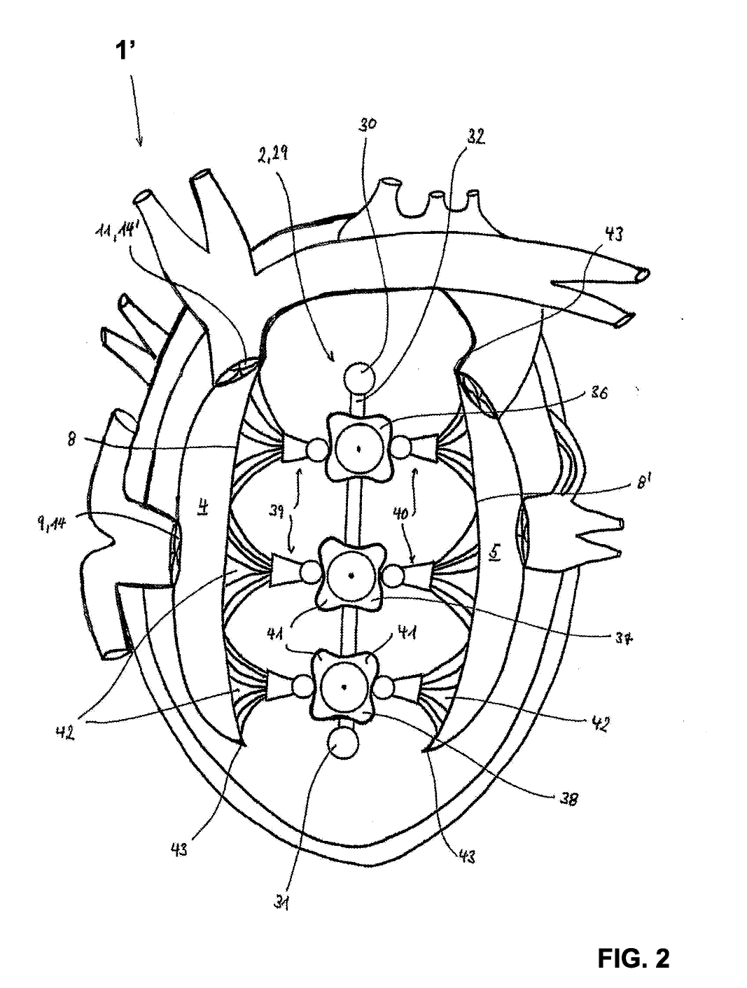

FIG. 2 illustrates another embodiment wherein the actuation device 2 of the heart 1′ illustrated therein is not formed by a pump 15 but through an actuation mechanism 29. Accordingly the heart 1′ according to FIG. 2 has no operating cavities 15, 16 and no compensation cavity 18.

The actuation mechanism 29 is instead formed from two motors 30, 31 and a worm gear 32, wherein the worm gear 32 does engage non illustrated gears 33, 34, 35 which in turn are respectively connected with a cam 36, 37, and 38 in a rigid manner. When the motors 30, 31 cause a rotation of the worm gear 32 the latter brings gears 33, 34, 35 into rotation, wherein eventually the cams 36, 37, 38 also start to rotate. Two respective tappets 39, 40 are arranged on the cams 36, 37, 38 which perform a lift stroke through the rotation of the respective associated cams 36, 37, 38. For this purpose the cams 36, 37, 38 are provided with four respective protrusions 41 which cause a lifting of the tappets 39 during rotation of the cams 36, 37, 38. Through the arrangement of the four protrusions 41 on each of the cams 36, 37, 38. The tappets 39, 40 perform a total of four lift strokes per one revolution of the cams 36, 37, 38.

The tappets 39, 40 are supported in a linear manner by a support device that is not illustrated so that they can only move in a straight line. At a side oriented away from the cams 36, the tappets 39, 40 are respectively provided with an expansion 42 which is connected with an associated flexible wall 8, 8′ at an end oriented away from the respective tappet 39, 40. This way a force transmitting connection between the tappets 39, 40 and the flexible walls 8, 8′ is established which eventually transposes the lift strokes of the tappets 39, 40 into lift strokes of the flexible walls 8, 8′. A complete lift stroke of the tappets 39, 40 from a low position in which a distance of the respective tappet 39, 40 from a rotation axis of an associated cam 36, 37, 38 is minimal to the a high position in which a distance of the respective tappet 39, 40 from the rotation axis of the associated cam is a maximum and back into the low position causes a respective transfer of the respective associated flexible wall 8, 8′ from the suction position into the pumping position and back into the suction position. The expansions 42 at the respective tappets 39, 40 are thus used for a homogeneous force transmission from the tappets 39, 40 to the flexible walls 8, 8′ so that the risk of a merely local deformation of the flexible walls 8, 8′ is not incurred.

Thus, a reversal of the flexible walls 8, 8′ from the pumping position into the suction position in the embodiment illustrated in FIG. 2 is caused by the flexible walls 8, 8′ itself. These are made from an elastic material and connected in a border portion 43 to the associated rigid wall 7, 7′. The flexible walls 8, 8′ are installed into the heart 1′ in their suction position wherein they are provided with a light preload which tends to pull the respective flexible walls 8, 8′ straight, this means to bring them into a flat shape. This is only prevented by the tappets 39, 40 which are in their low positions so that an axial force impacts the tappets 39, 40 which presses the tappets 39, 40 onto the respective associated cams 36, 37, 38. When the cams 36, 37, 38 are eventually rotated by the motors 30, 31 and in turn the tappets 39, 40 are transferred from their low position into their high position the flexible walls 8, 8′ in addition to its preload is deformed elastically which in turn further increases the described reset force. The motors 30, 31, however, counteract the reset force, so that the cams 36, 37 continue to rotate and lift the tappets 39, 40 against the reset force. When the tappets 39, 40 have eventually reached their high position, thus the flexible walls 8, 8′ are in their pumping position, the reset force of the flexible walls 8, 8′ causes the tappets 39, 40 not to lift off as soon as they are beyond their high point, this means they do not lose a contact to the cams 36, 37, 38 that continue to rotate but are pressed into valleys between the peaks 41 of the cams 36, 37, 38. The initially applied preload thus has the effect that a lift off of this type does not occur in the low position of the tappets 39, 40 either.

The arrangement of two tappets 39, 40 per cam 36, 37, 38 is particularly advantageous in the embodiment illustrated in FIG. 2, since both flexible walls 8, 8′ can be moved simultaneously this way. This movement is performed simultaneously in analogy to a natural heart, this means that all tappet 39, 40 are approximately simultaneously in their high position or low position, thus the flexible walls 8, 8′ accordingly are simultaneously in their suction position or in their pumping position.

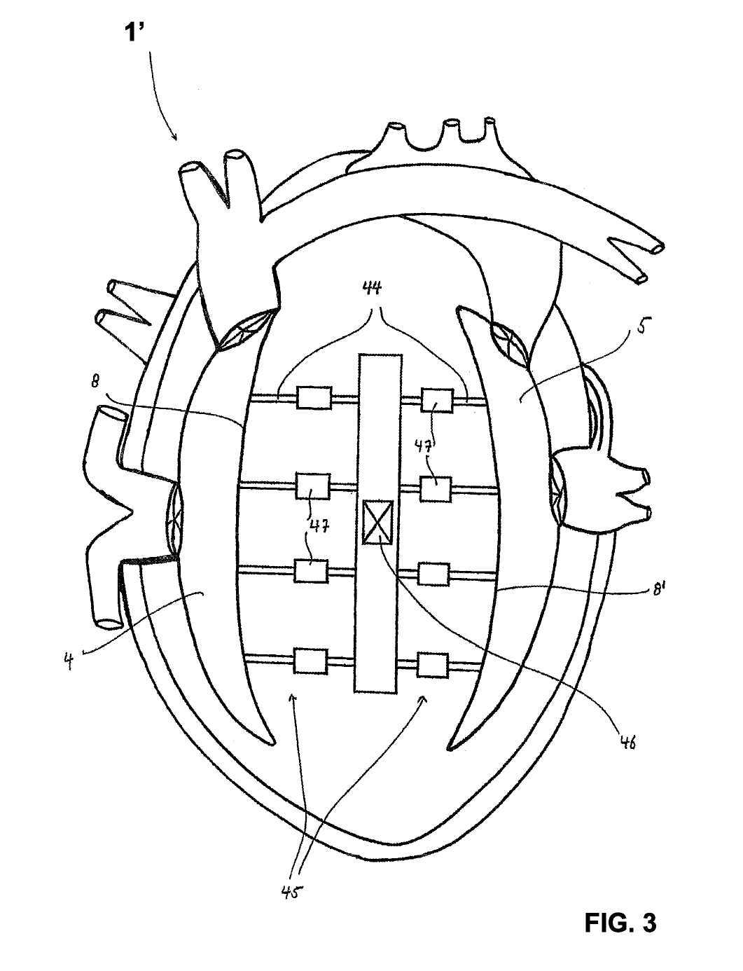

In another embodiment that is illustrated in FIG. 3, the deformation of the flexible walls 8, 8′ of the pumping chambers 4, 5 of a heart 1″ is caused by pistons 44 which respectively are elements of a piston/cylinder unit 45. In order to achieve an even force introduction into the walls 8, 8′ a plurality of pistons 44 is advantageously used in illustrated embodiment. This way locally limited deformations of the flexible walls 8, 8′ are counteracted which may lead to a formation of individual “dents” in which blood collects and then comes to a standstill which could lead to blood clotting.

The pistons 44 of the piston-cylinder units 45 are pressure loaded through a pump 46 which is part of the actuation device 2, wherein the pump 46 impacts a transmission fluid which is thus pressurized. This transmission fluid is arranged in cylinders 47 of the piston-cylinder unit 45 so that the cylinder 47 is loaded with the same pressure through an increase of a pressure level of the transmission fluid wherein a bottom side of a respective piston 44 arranged in a cylinder 47 is moved by this pressure from a low position into a high position. The piston-cylinder unit 45 accordingly exactly performs the function of the cams 36, 37, 38 and of the tappets 39, 40 of the embodiment according to FIG. 2. The remaining functions are identical. The transmission fluid is a fluid that is body friendly which does not pose any health hazards for the patient if the artificial heart 1′ is damaged. Though the heart 1″ is provided tight as a matter of principle even in case of internal leaks no fluid exchange can occur between the bodies of the patient of the heart 1″ a substantial damage to the heart 1″ could be caused by an accident so that transmission fluid exits.

Using piston-cylinder units 45 thus has the advantage that the piston cylinder units are respectively individually controllable so that a time offset lifting motion of individual pistons can be caused in a particularly simple manner. It is possible in particular that the two flexible walls 8, 8′ are controlled in a different manner, wherein the two flexible walls 8, 8′ always deform in the same manner when the heart 1′ deforms, in particular they deform simultaneously. By the same token it is feasible by time offset control to achieve a peristaltic pumping effect through the flexible walls 8, 8′.

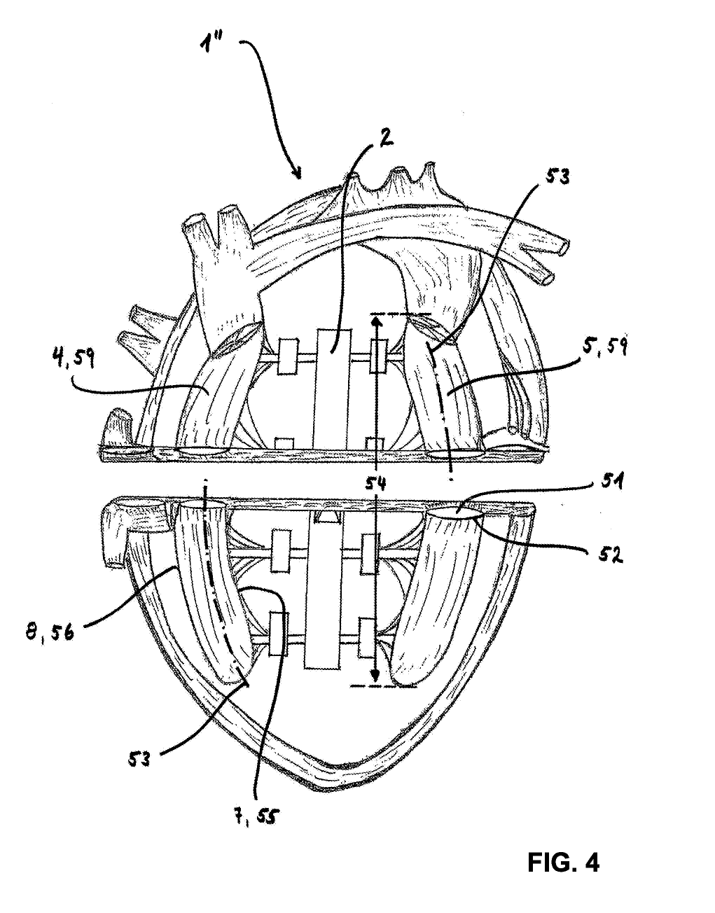

FIG. 4 illustrates a cross section through the artificial heart 1″ described supra which emphases the geometry of the pumping chambers 4, 5, namely the cross section indicates that a cross section surface 51 of the pumping chambers 4, 5 is enveloped by an ellipse. The pumping chambers 4, 5 furthermore include a centerline 53 represented by a dash dotted line which has a camber radius. The camber of the pumping chambers 4, 5 describes a circular arc in the illustrated embodiment. As an alternative it is also conceivable that a camber radius of a center line 53 of a respective pumping chamber 4, 5 is variable over the length of the pumping chamber. In the illustrated embodiment the camber radius of the center line 53 is 20 cm. The center line 53 can be interpreted as a connecting line of all centers of gravity of all cross sections of the respective pumping chambers 4, 5.

The respective pumping chambers 4, 5 of the heart 1″ have a length 54 of approximately 10 cm wherein the length 54 of a pumping chamber 4, 5 is defined as a connecting line between the centers of gravity of terminal cross sections of the respective pumping chamber 4, 5 wherein the terminal cross sections of a pumping chamber 4, 5 are defined as cross sections through the pumping chamber 4, 5 which extend orthogonal to the center line 53 and have a maximum offset from one another.

A semi major axis 57 of the ellipse 52 that is not illustrated in FIG. 4 has a length of 1.8 cm wherein the length of the semi minor axis 58 which is also not illustrated in FIG. 4 is 1.8 cm. Therefore the volume of the pumping chamber 4, 5 is approximated as V=80 cm³, wherein the volume of a circular arc with an elliptical cross section is used as an approximation.

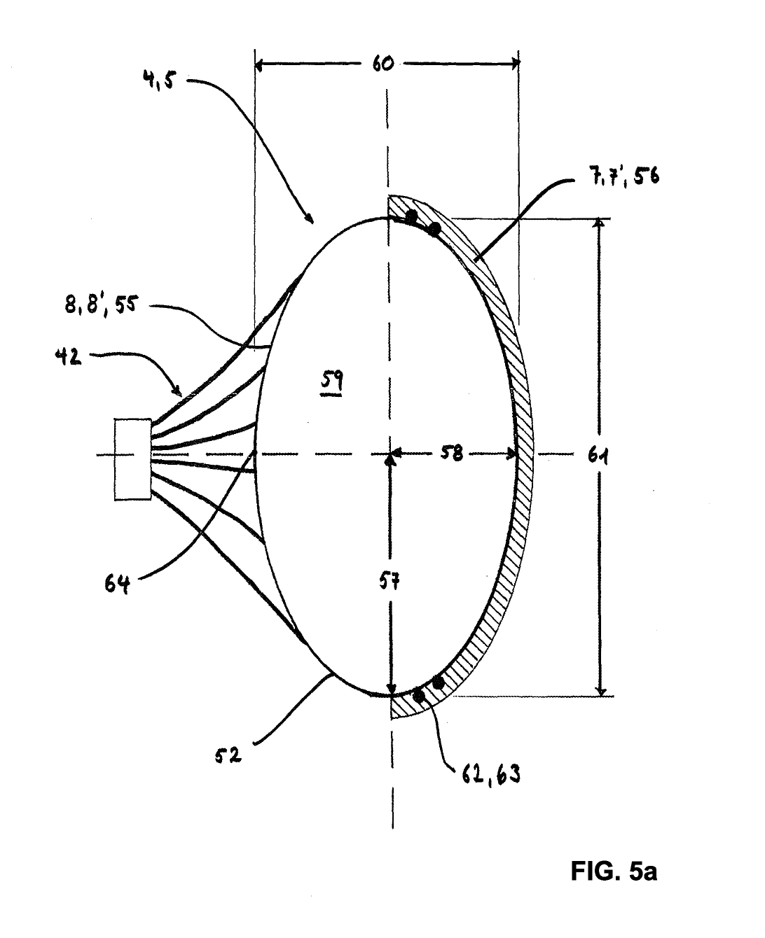

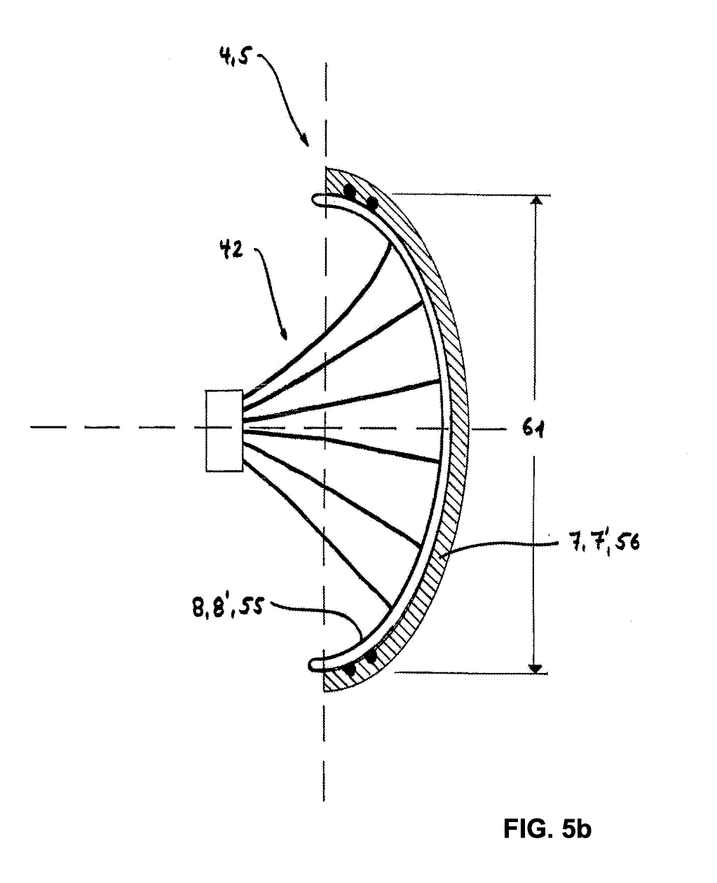

The pumping chambers 4, 5 respectively have an elastic envelope 55 which is formed by the flexible walls 8, 8′. The elastic envelope 55 defines an inner cavity 59 of the pumping chambers 4, 5. A support portion of the elastic envelope 55 of the respective pumping chambers 4, 5 which support portion is oriented away from the actuation device 2 is coupled through a rigid support element 56 and thus stiffened. The configuration of the pumping chambers 4, 5 with respect to the subdivision into rigid walls 7, 7′ and flexible walls 8, 8′ can be easily derived from FIGS. 5a, and 5b described infra.

The figures illustrate a cross section through one of the pumping chambers 4, 5 of the heart 1″, wherein the pumping chambers 4, 5 are illustrated in FIG. 5a is in their suction positions (maximum volume) and in their pressure position (minimum volume) in FIG. 5b. An edge of a cross section has a shape of an ellipse 52 wherein a semi major axis 57 has a length of 1.8 cm and a semi minor axis 58 has a length of 1.4 cm. Accordingly a height 60 of the respective pumping chambers 4, 5 is 2.8 cm and a width 61 is 3.6 cm. An area of the cross section through the pumping chamber 4, 5 accordingly computes as A=π×a×b=7.9 cm².

From FIG. 5 it is apparent that the inner cavity 59 of the pumping chambers 4, 5 is completely enveloped by the elastic envelope 55 and therefore defined by the elastic envelope 55. Half the circumference of the elastic envelope 55 is enveloped by a rigid support element 56 and connected with the rigid support element 56 through form locking so that a force can be transferred. The support element 56 has a U shaped cross section which envelops the elastic envelope 55. The support element 56 thus forms a support portion for the elastic envelope 55 which stiffens the elastic envelope 55. In connection with the support element 56 the elastic envelope 55 is therefore not flexible anymore but rigid. Accordingly the support portion of the elastic envelope 55 forms the “rigid wall 7′, 7” wherein the remaining portion in which the elastic envelope 55 can deform freely forms the flexible walls 8, 8′ of the respective pumping chamber. The support element 56 extends over the entire length of the pumping chambers 4, 5.

The form locking between the elastic envelope 55 and the support element 56 is configured through form locking by interaction of a groove and a key. The support element 56 thus includes a total of four grooves 62 which extend parallel to the center line of the respective pumping chamber 4, 5 over the entire length of the pumping chambers 4, 5. The grooves 62 are advantageously configured undercut. The elastic envelope 55 includes keys 63 that correspond to the grooves 62 and engage the grooves 62 precisely fitting. In addition to the connection of the elastic envelope 55 with the support element 56 a full surface glue connection can be advantageously provided.

Based on the suction position of the flexible wall 8, 8′ illustrated in FIG. 5 a “lowest location” 64 of the flexible wall 8, 8′, this means the location of the cross section through the pumping chamber 4, 5 which is closest to the actuation device 2 has to be deflected by the height 60 of the pumping chamber 4, 5 so that the volume of the pumping chamber 4, 5 in its pumping position is minimal, preferably equal to zero.

The latter pumping position is illustrated in FIG. 5b as described. Herein it is in particular visible quite well that the elastic envelope 55 is pressed completely against the support element 56 which reduces the volume of the inner cavity 59 of the pumping chamber 4, 5 approximately to zero. The blood disposed in the pumping chamber 4, 5 is therefore displaced completely.

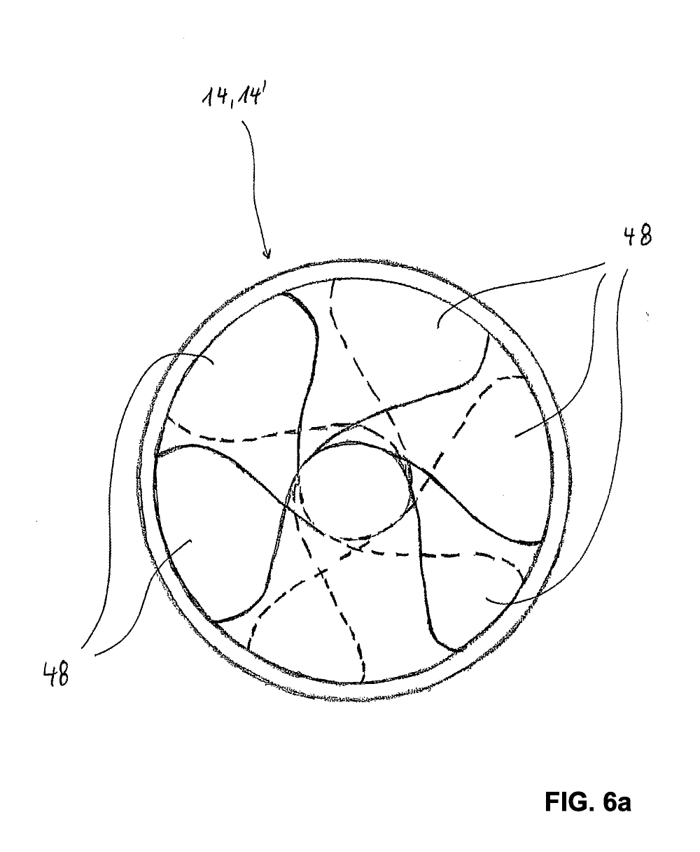

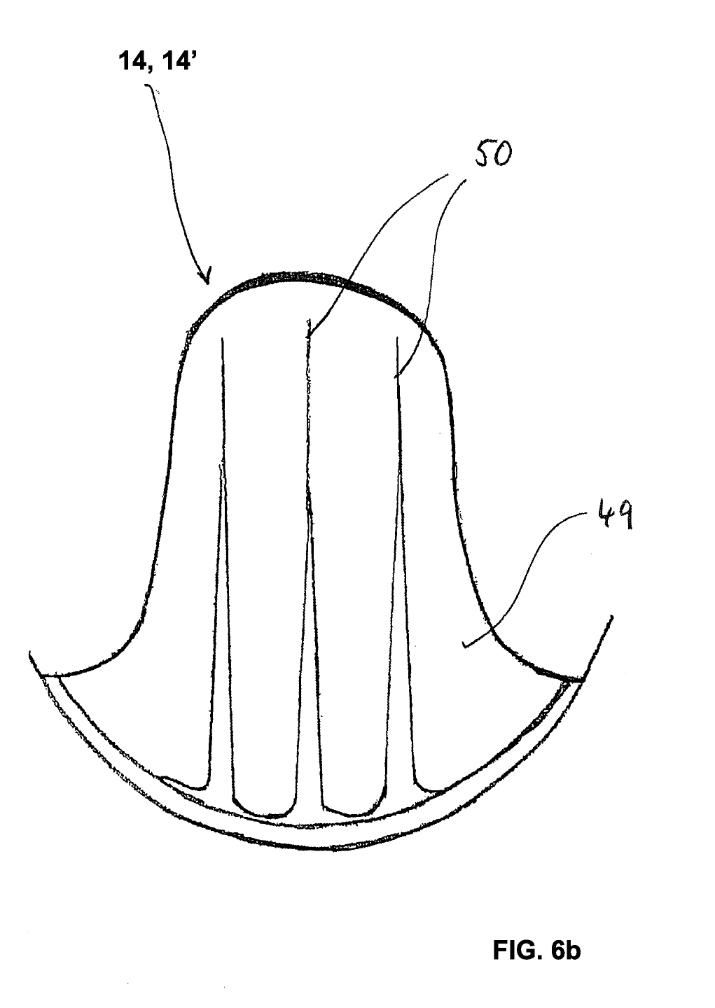

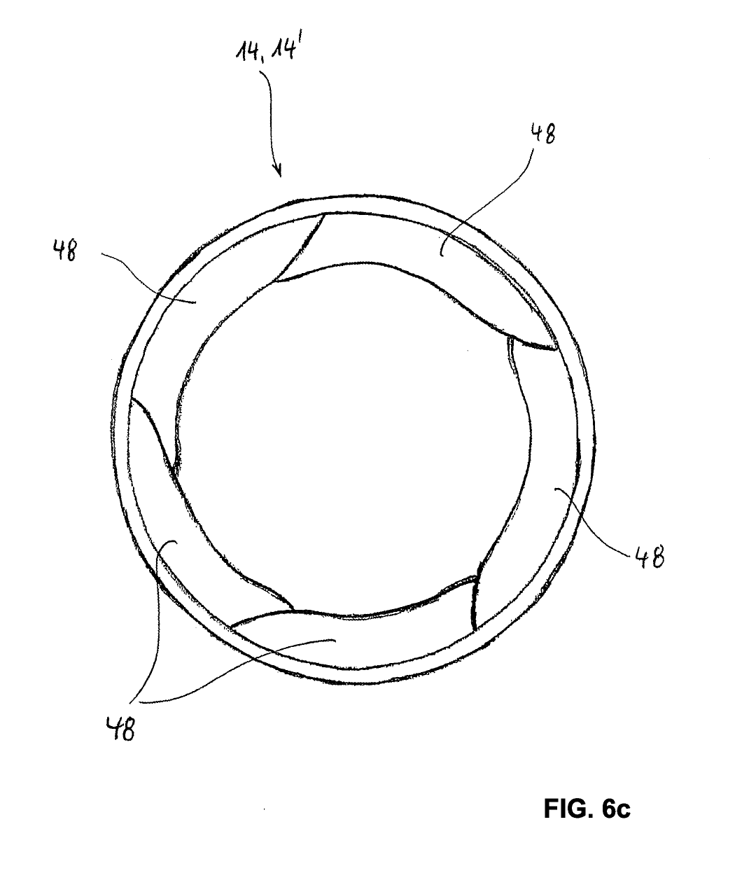

In FIGS. 6a-c different representations of a flap element 14, 15 are illustrated which takes over the function of a non return valve and is respectively arranged at the inlet openings 9, 10 and the outlet openings 11, 12.

FIG. 6a illustrates a top view of a flap element 14, 14′ arranged in a closed position, wherein individual flap segments 48 of the flap element 14, 14′ overlap in the closed position and close an opening in this manner. In the illustrated configuration a fluid flow cannot flow through the flap elements 14, 14′ in a direction orthogonal to the drawing plane. The individual flap segments 48 are thus arranged along an opening cross section wherein the illustrated embodiment provides a total of 5 flap segments 48 which are arranged in the opening cross section evenly distributed about the circumference.

Each individual flap segment 48 has approximately a shape of a regular parabola as apparent from FIG. 6b. The flap segment 48 includes a flat segment blade 49 and three reinforcement elements 50 which prevent a flapping through of the flap element 48 beyond the closed position. Due to these reinforcement elements 50 the flap elements 48 can only be opened in one direction in which the fluid flow shall be released.

An open position of the flap element 14, 14′ can be derived from FIG. 6c. The individual flap segments 48 are thus pivoted up so that they are substantially oriented parallel to the fluid flow. The opening is released from this position. When the fluid flow is inverted in a direction in which the flap elements 14, 14′ shall close, the flap segments 48 are automatically forced into their closed position through a movement of the fluid flow from the open position so that the functional principle of a no return valve is simulated.

### REFERENCE NUMERALS AND DESIGNATIONS

1,1′,1″ heart
2 actuation device
3 energy storage device
4 pumping chamber
5 pumping chamber
6 envelope
7, 7′ rigid wall
8,8′ flexible wall
9 inlet opening
10 inlet opening
11 outlet opening
12 outlet opening
13 wall
14, 14′ flap element
15 pump
16 operating cavity
17 operating cavity
18 compensation cavity
19 conduit
20 conduit
21 conduit
22 conduit
23 conduit
24 conduit
25 connection cavity
26 connection cavity
27, 27′ conduit
28, 28′ conduit
28′, 28′″ conduit
29 actuation mechanism
30 motor
31 motor
32 worm gear
33 gear
34 gear
35 gear
36 cam
37 cam
38 cam
39 tappet
40 tappet
41 rise
42 expansion
43 border portion
44 piston
45 piston-cylinder unit
46 pump
47 cylinder
48 flap segment
49 segment blade
50 reinforcement element
51 cross section area
52 ellipse
53 center line
54 length
55 elastic envelope
56 support element
57 semi major axis
58 semi minor axis
59 interior cavity
60 height
61 width
62 groove
63 key
64 lowest spot

What is claimed is:

1. An artificial heart, simulating a human heart, comprising:
an energy storage device;
an actuation device; and
two pumping chambers, each including an inlet opening and an outlet opening,
wherein each pumping chamber is defined by at least one flexible wall and one rigid wall,
wherein a volume of each pumping chamber is variable by deforming the at least one flexible wall through the actuation device,
wherein the at least one flexible wall, starting from a suction position in which a volume of the respective pumping chamber is at a maximum, is transferable through the actuation device to a pumping position in which the volume of the respective pumping chamber is at a minimum,
wherein the inlet opening and the outlet opening of each pumping chamber each have a non-return valve and the non-return valve is reciprocally transferable between an open position and a closed position, whereby a directed flow of blood from the respective inlet opening to the respective outlet opening is provided,
wherein the actuation device is located between the two pumping chambers,
wherein the two pumping chambers each have an elongated shape,
wherein a centerline of each pumping chamber is cambered and has a radius of camber between 10 cm and 40 cm, and
wherein a pumping chamber cross-section orthogonal to the centerline of the respective pumping chamber has a form of an ellipse, wherein a ratio between a semi major axis and a semi minor axis of the ellipse is between 1.0 and 2.0.

2. The artificial heart according to claim 1,
wherein an inner cavity of a respective pumping chamber is defined by an elastic envelope,
wherein the elastic envelope forms the rigid wall of the pumping chamber in a support portion that is oriented away from the actuation device, over an entire length of the pumping chamber, through cooperation with a rigid support element, and
wherein the elastic envelope forms the flexible wall of the pumping chamber outside of the support portion,
wherein half of a circumference of the elastic envelope is advantageously enveloped by the rigid support element in a cross section through the elastic envelope orthogonal to the center line of the respective pumping chamber.

3. The artificial heart according to claim 2,
wherein the elastic envelope and the rigid support element are connected with one another through form locking in a force transferring manner, and
wherein the support element includes at least one groove over its entire length in which a corresponding key of the elastic envelope engages.

4. The artificial heart according to claim 1,
wherein a second envelope completely envelops both pumping chambers and the actuation device, and
wherein the energy storage device is furthermore advantageously arranged in an interior of the second envelope.

5. The artificial heart according to claim 1, wherein for one of the two pumping chambers an arrangement of the associated outlet opening corresponds to a natural arrangement of a lung artery of a right heart chamber of a human heart.

6. The artificial heart according to claim 1, wherein for one of the two pumping chambers an arrangement of the associated outlet opening corresponds to a natural arrangement of an aorta of a left heart chamber of a human heart.

7. The artificial heart according to claim 1,
wherein a connecting cavity is arranged at one of the two pumping chambers on a side of the associated inlet opening which side is oriented away from the pumping chamber, and
wherein two conduits conducting a blood flow lead into the connecting cavity.

8. The artificial heart according to claim 1,
wherein a connecting cavity is arranged at one of the two pumping chambers on a side of the associated inlet opening which side is oriented away from the pumping chamber,
wherein four conduits lead in the connecting cavity to conduct the blood flow.

9. The artificial heart according to claim 1,
wherein at least one flexible wall of at least one pumping chamber is moved by at least one transmission device,
wherein the at least one transmission device is driven by the actuation device and the at least one transmission device is formed by a piston-cylinder unit,
wherein a piston of the piston cylinder unit is connected with the flexible wall and a cylinder is loadable with an actuation pressure through the actuation device, and
wherein the actuation device includes a pump.

10. The artificial heart according to claim 16, wherein the tappet performs a plurality of strokes during a single revolution of the cam.

11. The artificial heart according to claim 16, wherein the cam cooperates with a plurality of tappets advantageously with two tappets.

12. The artificial heart according to claim 1,
wherein at least one flexible wall of at least one pumping chamber is moveable through a hydraulic pressure,
wherein the flexible wall is loadable with a transmission fluid advantageously on a side that is oriented away from the pumping chamber,
wherein the transmission fluid is loadable with a pressure through the actuation device, and
wherein the actuation device includes a pump and the hydraulic pressure advantageously impacts an entire surface of a side of the respectively associated flexible wall, which side is oriented away from the pumping chamber.

13. The artificial heart according to claim 1,
wherein at least one non return valve is formed by a flap element, and
wherein the flap element includes a plurality of flap segments through which the flap element is closed when a blood flow flows in a closing direction and vice versa when the blood flow flows in an opening direction the flap element is released.

14. The artificial heart according to claim 1,
wherein at least one of the flexible walls includes individual portions which are deformable in a time offset manner through the actuation device during a transfer of the entire flexible wall from the suction position into the pumping position.

15. The artificial heart according to claim 1, wherein the at least one non return valve is removably attached at the respective inlet opening or outlet opening through a clip system providing a form locking connection.

16. The artificial heart according to claim 1,
wherein at least one flexible wall of at least one pumping chamber is moved by at least one transmission device,
wherein the at least one transmission device is driven by the actuation device and the at least one transmission device is formed by a cam-tappet unit,
wherein a cam of the cam-tappet unit is drivable by the actuation device and a tappet is connected with the flexible wall.

17. An artificial heart, simulating a human heart, comprising:
an energy storage device;
an actuation device; and
two pumping chambers, each including an inlet opening and an outlet opening,
wherein each pumping chamber is defined by at least one flexible wall and one rigid wall,
wherein a volume of each pumping chamber is variable by deforming the at least one flexible wall through the actuation device,
wherein the at least one flexible wall, starting from a suction position in which a volume of the respective pumping chamber is at a maximum, is transferable through the actuation device to a pumping position in which the volume of the respective pumping chamber is at a minimum,
wherein the inlet opening and the outlet opening of each pumping chamber each have a non-return valve and the non-return valve is reciprocally transferable between an open position and a closed position, whereby a directed flow of blood from the respective inlet opening to the respective outlet opening is provided,
wherein the actuation device is located between the two pumping chambers,
wherein the two pumping chambers each have an elongated shape,
wherein a centerline of each pumping chamber is cambered and has a radius of camber between 15 cm and 30 cm, and
wherein a pumping chamber cross-section orthogonal to the centerline of the respective pumping chamber has a form of an ellipse, wherein a ratio between a semi major axis and a semi minor axis of the ellipse is between 1.1 and 1.5.
